(19) United States
(12) Patent Application Publication
(10) Pub. No.: US 2005/0186298 A1
(43) Pub. Date: Aug. 25, 2005

(54) Methods of stabilizing carotinoid-containing biomasses

(76) Inventors: Wilhelm Johannisbauer, Erkrath (DE); Andreas Sander, Illertissen (DE); Eike Ulf Mahnke, Ratingen (DE)

Correspondence Address: COGNIS CORPORATION;PATENT DEPARTMENT, 300 BROOKSIDE AVENUE, AMBLER, PA, 19002, US

(21) Appl. No.: 11/047,170

(22) Filed: Jan. 31, 2005

(30) Foreign Application Priority Data
Jan. 31, 2004 (DE) 10 2004 004 925.4

(51) Int. Cl. C12P 23/00; A61K 31/355; A61K 31/12

(52) U.S. Cl. 424/780; 514/458; 514/548; 514/690; 435/67

(57) ABSTRACT

The use of tocopherol mixtures for stabilizing carotinoid-containing biomasses and biomass preparations, more particularly astaxanthin-containing biomasses and biomass preparations, and to a process for stabilizing such biomasses and biomass preparations. The tocopherol mixture used preferably contains α- and β- and γ- and δ-tocopherol. The invention also relates to carotinoid-containing biomass containing 0.1 to 10% by weight of tocopherol mixtures, based on the weight of the dry biomass.

### BACKGROUND OF THE INVENTION

Carotinoids and carotinoid derivatives are oxidation-sensitive substances which are used inter alia for coloring foods and in cosmetic or pharmaceutical preparations. In the food sector for example, salmon is given its characteristic pink color by addition of the carotinoid astaxanthin to the fish food. The astaxanthin may either be of synthetic origin or may be obtained from natural sources (Cyanotech, Mera Pharmaceuticals). Astaxanthin is a carotinoid which may be obtained from microalgae and which in recent years has become known through its increasing use in aquaculture for coloring trout and salmon meat. This carotinoid has many other applications. For example, it is used inter alia as a radical quencher, hormone precursor, growth promoter, photoprotector or immunostimulant. One of its most important functions is as an antioxidant because it has ten times the antioxidative capacity of other carotinoids, such as β-carotene for example.

In view of their high sensitivity to oxygen, heat and light, carotinoids such as astaxanthin can easily be destroyed in storage. Degradation can even occur if the carotinoid is embedded in a matrix of other substances, such as lipids and proteins for example. In order to avoid degradation, antioxidants have to be added to and incorporated in the carotinoid-containing preparation. Hitherto, synthetic substances, such as ethoxyquin (1,2-dihydro-6-ethoxy-2,2,4-trimehylquinoline) or BHT (2,6-di-t-butyl-4-methylphenol), have been used as stabilizers for this purpose. Mixtures of different amines and amides, as disclosed in International application WO 0146355, or the urea described in International application WO 0001249 are also suitable for stabilization. However, these stabilizers are increasingly producing side effects and triggering allergic reactions, so that there is a need for equally effective, but more compatible stabilizers.

95% of the astaxanthin currently used on the market is synthetically produced. Since the demand for natural products and their use in foods, pharmaceutical and cosmetic preparations is steadily increasing, astaxanthin has recently been increasingly obtained from aquatic sources, the microalgae Haematococcus pluvialis being the natural source with the highest content of astaxanthin. This microalgae can be used as dry biomass. Thus, International application WO 99/13855 discloses formulations containing extracts of Haematococcus pluvialis for the treatment of rough, dry skin.

Even in the form of dry carotinoid-containing biomass, astaxanthin is vulnerable to decomposition, particularly at elevated temperature, so that the storage time is very limited despite drying.

Accordingly, the problem addressed by the present invention was to stabilize carotinoid-containing biomass in a physiologically and toxicologically safe form against degradation of the carotinoid so that it could be stored at room temperature or elevated temperature.

### SUMMARY OF THE INVENTION

This invention relates generally to cosmetic and pharmaceutical preparations and to animal feeds and foods for human consumption. More particularly, the invention relates to the use of tocopherol mixtures for stabilizing carotinoid-containing biomass and to the stabilized biomass containing tocopherol mixtures.

The present invention relates to the use of tocopherol mixtures for stabilizing carotinoid-containing biomass and biomass preparations, more particularly for stabilizing astaxanthin-containing biomass.

It has surprisingly been found that natural tocopherol mixtures are suitable, even in dry systems, for stabilizing carotinoid-containing biomass and astaxanthin and astaxanthin esters produced biosynthetically with the green algae Haematococcus pluvialis. The incorporation of the tocopherol mixtures enables the storage life of solid carotinoid-containing biomasses and biomass preparations to be considerably lengthened, even at room temperature and elevated temperatures. The careful incorporation of the antioxidants in the algal biomass suspended in water in a process combined with cell digestion results in a product which is completely free from synthetic additives and which can be stored far better than unstabilized dry biomass. Accordingly, the invention provides a readily “bioavailable” product free from synthetic stabilizers for use in animal feeds, cosmetics and pharmaceutical preparations. Tocopherol mixtures are distinguished by very high compatibility.

### DETAILED DESCRIPTION OF THE INVENTION

Tocopherols are understood to be chroman-6-ols (3,4-dihydro-2H-1benzopyran-6-ols) substituted in the 2 position by 4,8,12-trimethyltridecyl groups. They correspond to the following formula (I): 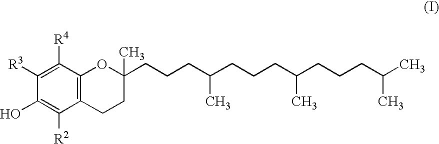
in which R², R³ and R⁴ independently of one another represent hydrogen or a methyl group. Tocopherols are also known as bioquinones. Typical examples are the plastiquinones, tocopherol quinones, ubiquinones, boviquinones, K vitamins and menaquinones (for example 2-methyl-1,4-naphthaoquinones). The quinones from the vitamin E series, i.e. α-, β-, γ-, δ- and ε-tocopherol (the last of these still having the original unsaturated prenyl side chain), are preferably used.

The particularly preferred form in which the tocopherol mixtures are used contains α- and β- and γ- and δ-tocopherol, optionally in combination with emulsifiers. Tests with various tocopherols have shown that the natural mixture containing 5 to 20% by weight and preferably 12 to 17% by weight of α-tocopherol, 0.01 to 2% by weight and preferably 0.1 to 0.5% by weight of β-tocopherol, 30 to 80% by weight and preferably 50 to 70% by weight of γ-tocopherol and 10 to 40% by weight and preferably 20 to 30% by weight of δ-tocopherol (obtainable under the name of Covi-ox® from Cognis Deutschland GmbH & Co. KG, Düsseldorf) has the best stabilizing effect.

The carotinoid-containing biomass is preferably obtained from algae, Haematococcus pluvialis preferably being used as the main starting material for production from aquatic sources because it has a high astaxanthin content.

Carotinoids, such as astaxanthin, are stabilized against oxidative degradation in the digested algal biomass as matrix substance by addition of the tocopherol mixtures. The tocopherol mixtures are used in quantities of 0.1 to 10% by weight, preferably in quantities of 0.2 to 5% by weight and more preferably in quantities of 0.3 to 2% by weight, based on the weight of the dry biomass.

Besides the surface-active substances occurring naturally in the algae, additional emulsifiers may also be added to the system for improving effectiveness.

The tocopherol mixtures are incorporated in the aqueous biomass suspension in a parallel process combined with cell digestion or in a second stage immediately after digestion. If the biomass suspension is mechanically dewatered before drying, it is important to ensure that no stabilizer is removed with the water.

### EXAMPLES

Haematococcus pluvialis biomass with an astaxanthin content of 4.5% by weight, based on the weight of the dry biomass, and the two stabilizers Controx® KS (Cognis Deutschland GmbH & Co. KG, Düsseldorf) and Controx® VP (Cognis Deutschland GmbH & Co. KG, Düsseldorf) were used to demonstrate the stabilizing effect. Controx® KS contains 80% by weight of mixed tocopherols and 20% by weight of citric acid mono- and diglycerides. Controx® VP contains 25% by weight of mixed tocopherols, 10% of citric acid mono- and diglycerides, 25% by weight of ascorbyl palmitate and 40% by weight of lecithin (%=wt. %).

Based on dry matter, the samples were intensively dispersed with 10% by weight of one of the two mixed tocopherol preparations, and as reference with no stabilizer, in aqueous suspension and then freeze-dried.

The dry samples were incubated for 38 days at −20° C., 20° C. and 40° C. in non-hermetically sealed glass containers in order to simulate the storage process. Determination of the astaxanthin contents by solvent extraction and high-performance liquid chromatography leads to the results set out in Table 1 below.

It can seen from these results that storage in the frozen state is possible, even without stabilizer, although the astaxanthin in the unstabilized samples is completely degraded over the storage period both at room temperature and at elevated temperature. Whereas the stabilizers are comparable in their effectiveness at room temperature, the product with the higher tocopherol content is clearly superior at elevated temperature.

TABLE 1
Astaxanthin content of differently stabilized biomass after 38 days' storage

| Temperature [° C.] | Addition | Astaxanthin content [% by wt.] |
|---|---|---|
| −20 | None | 4.5 |
| −20 | Controx ® KS | 4.4 |
| −20 | Controx ® VP | 4.5 |
| 20 | None | 0.0 |
| 20 | Controx ® KS | 3.5 |
| 20 | Controx ® VP | 3.4 |
| 40 | None | 0.0 |
| 40 | Controx ® KS | 2.3 |
| 40 | Controx ® VP | 1.7 |

1. A method of stabilizing a carotinoid-containing biomass, said method comprising:
(a) providing a carotinoid-containing biomass; and
(b) combining the carotinoid-containing biomass and a mixture of two or more tocopherols.

2. The method according to claim 1, wherein the carotinoid-containing biomass comprises astaxanthin.

3. The method according to claim 1, wherein the mixture comprises α-, β- and γ-tocopherol.

4. The method according to claim 1, wherein the carotinoid-containing biomass comprises astaxanthin, and wherein the mixture comprises α-, β- and γ-tocopherol.

5. The method according to claim 1, wherein the mixture is combined with the biomass in an amount of from 0.1 to 10% by weight, based on the dry weight of the biomass.

6. The method according to claim 2, wherein the mixture is combined with the biomass in an amount of from 0.1 to 10% by weight, based on the dry weight of the biomass.

7. The method according to claim 3, wherein the mixture is combined with the biomass in an amount of from 0.1 to 10% by weight, based on the dry weight of the biomass.

8. The method according to claim 4, wherein the mixture is combined with the biomass in an amount of from 0.1 to 10% by weight, based on the dry weight of the biomass.

9. The method according to claim 1, wherein the biomass is derived from Haematococcus pluvialis.

10. A method of stabilizing an astaxanthin-containing biomass, said method comprising:
(a) providing an astaxanthin-containing biomass; and
(b) combining the astaxanthin-containing biomass and a tocopherol mixture comprising α-, β- and γ-tocopherols; wherein the mixture is combined with the biomass in an amount of from 0.1 to 10% by weight, based on the dry weight of the biomass.

11. A biomass comprising at least one carotinoid and a mixture of two or more tocopherols, wherein the mixture is present in an amount of from 0.1 to 10% by weight, based on the dry weight of the biomass.

12. The biomass according to claim 11, wherein the at least one carotinoid comprises astaxanthin.

13. The biomass according to claim 11, wherein the mixture comprises α-, β- and γ-tocopherol.

14. The biomass according to claim 11, wherein the at least one carotinoid comprises astaxanthin, and wherein the mixture comprises α-, β- and γ-tocopherol.

15. The biomass according to claim 11, wherein the biomass is derived from Haematococcus pluvialis.
